(19) United States
(12) Patent Application Publication
(10) Pub. No.: US 2003/0162837 A1
(43) Pub. Date: Aug. 28, 2003

(54) Carboxyfullerenes and methods of use thereof

(76) Inventors: Laura L. Dugan, St. Louis, MO (US); Eva G. Lovett, University City, MO (US); Kevin L. Quick, Florissant, MO (US); Joshua I. Hardt, Belleville, IL (US)

Correspondence Address: Paul A Lesko Esq, Thompson Coburn LLP, One Firstar Plaza, St Louis, MO, 63101, US

(21) Appl. No.: 10/083,283

(22) Filed: Feb. 23, 2002

(51) Int. Cl. A61K 31/19

(52) U.S. Cl. 514/574

(57) ABSTRACT

The instant invention is a method for increasing a metazoan's lifespan, comprising administering a carboxylated derivative of a C₆₀ fullerene. The instant invention further is a process for extending a metazoan's lifespan by administering a superoxide dismutase-mimetic as well as a composition comprising a superoxide dismutase-mimetic. Further, the instant invention comprises a pharmaceutical composition comprising carboxyfullerenes having x pairs of adjacent carbon atoms bonded to two carbons of the C₆₀ sphere wherein said adjacent carbon atom is further bonded to two groups of the general formula —COOH and —R, wherein R is independently selected from the group consisting of —COOH and —H, and wherein x is at least 1.

A further embodiment is a non-metal containing composition which can catalytically eliminate two biologically reactive species.

Another embodiment is a method of enhancing elimination of reactive oxygen species in eukaryotic cells by contacting cells with a superoxide dismutase mimetic.

### STATEMENT REGARDING FEDERALLY SPONSORED RESEARCH OR DEVELOPMENT.

This invention was made partially with government support under grant number NS37688 awarded by the National Institutes of Health. The government has certain rights to this invention.

### BACKGROUND OF THE INVENTION

1. Field of the Invention

This invention relates generally to a method for prolonging the length or duration of the expected lifespan (referred to alternately as “longevity”) of metazoans or in metazoan cells, and more particularly, to a method of extending a metazoan's lifespan by administering a composition comprising a therapeutically effective amount of an antioxidant.

2. Related Art

Methods of enhancing the overall health and longevity of humans and their companions has been a very active area of research. Current thinking in the field suggests that calorie restriction may help to extend the lifespan of metazoans.

Given the conserved nature of cellular or developmental processes across metazoans, a number of model organisms have been employed to study longevity including C. Elegans and D. Melanogaster.

For example, the genetic analysis of C. elegans has revealed several genes involved in lifespan determination. Mutations in Daf-2 (the insulin receptor) and Clk-1 (“Clock 1”, a gene affecting many aspects of developmental and behavioral timing) have been shown to extend the lifespan of adults. However, Clk-1 mutants have a higher mortality rate in early life. At later stages of development, the Clk-1 mutants show an increase in longevity, perhaps by selecting for long-lived individuals in early life. The Clk-1 longevity phenotype is abolished by mutations in the gene encoding catalase, which is involved in superoxide/free radical metabolism. Additionally, elimination of coenzyme Q in C. elegans diet has been shown to extend lifespan.

C. elegans harboring mutations in the Eat gene have also shown an increased longevity, but exhibit decreased food intake and slowed metabolism. The enhanced longevity associated with this mutation has been attributed to calorie restriction, which has been shown to also increase longevity in metazoans.

In Drosophila, superoxide dismutase (SOD) and catalase over expression increased the lifespan of fruit flies by 35%. Mutations also in the Methuselah gene (“Mth”) have been shown to increase lifespan by 20%. The function of Mth, a G-protein coupled receptor, is not known, but mutants have shown an increased resistance to paraquat (a superoxide radical injury inducing agent) toxicity, suggesting it may be a stress-response gene.

Calorie restriction (CR) has been shown to increase lifespan by 25-35% in all animals studied to date (mice, rats, several species of monkeys, dogs, humans, as well as non-metazoan species such as spiders, Nematodes, and Drosophila. (NB: All animals are metazoans.) However, caloric intake needs to reduced by as much as 30-40% to achieve robust effects on longevity. Ongoing studies in rhesus and squirrel monkeys at the National Institute of Aging (“NIA”) (Roth et al., Eur. J. Clin. Nutr. S:15, 2000) found biochemical changes in calorie restricted monkeys similar to changes reported in rodents thereby supporting the universal nature of calorie restriction on biochemical processes across vertebrate species.

Recently, 2-deoxyglucose has been used to produce calorie restriction without limiting oral intake. Animals treated with 2-deoxyglucose have lowered body temperature and decreased plasma insulin levels, similar to changes observed in calorie-restricted animals (Roth et al., Ann. NY Acad. Sci., 928: 305, 2001). While scientific studies on the effect of 2-deoxyglucose on longevity have not been completed, a recent editorial in Science (Feb. 8, 2002) quoted the principal investigator of these studies (George Roth, NIA) as saying that one of his monkeys treated with 2-deoxyglucose lived 38 months instead of the mean survival of 25 months. However, such a claim is not scientifically supported given the small sample size. No comment was made on the age of the longest-lived monkeys in the control populations.

Increases of up to 20% in the expected lifespan of mice has been shown through growth factor deprivation, either through genetic manipulation or the administration of growth factor antagonists. Unfortunately, dwarfism is a side effect of growth factor deprivation. In humans, dwarfism, or late-life growth hormone deficiency, appears to reduce longevity, further confusing the issue of whether growth factor deprivation is effective as a means for increasing the duration of the expected lifespan.

Several papers have indicated that deprenyl (a selective monoamine oxidase (MAO) B inhibitor used to treat Parkinson's disease) increases the lifespan of many species. (See, e.g. Knoll, Mech Ageing Dev. 46:237, 1988). In one study, chronic treatment of rats with deprenyl from age 96 weeks through the end of life “enhanced survival”. Control rats lived 147+/−1 weeks, whereas the deprenyl-treated rats lived 198+/−2 weeks. However, the expected mean lifespan for these rats, clearly stated in the paper, was 182 weeks, so the control group in this study appears to have had early mortality. Other studies from these laboratories selected for high-performing rat, which were then enrolled in the deprenyl longevity studies, thereby potentially artificially skewing the results.

A second study used Fisher 344 rats (Kitani et al., Life Sci 52:281, 1993), initiating deprenyl treatment at 18 months of age. The mean survival of the controls was 28 months, and of the treated animals was 30 months, showing an increase in longevity of 7%. However, these results were shown to be not statistically significant.

In contrast, another study in Fisher 344 rats with the same dose of deprenyl (Carillo et al, Life Sci 67:2539, 2000), observed greater mortality and shortened lifespan in the deprenyl-treated animals. Furthermore, a study from the NIA failed to show any survival benefit in C57B6 mice given chronic deprenyl treatment starting at 18 months of age (Ingram et al., Neurobiol Aging 14:431, 1993). Likewise, a controlled study of deprenyl in Drosophila did not show an increase in lifespan (Jordens et al., Neurochem Res 24:227, 1999).

Human trials of deprenyl likewise show conflicting results regarding longevity. An “open, uncontrolled” trial of deprenyl in Parkinson's patients showed an increase survival at 9 years (Birkmayer et al., J Neural Transm. 64:113, 1985), although other studies have suggested increased mortality in PD patients taking deprenyl, especially in conjunction with L-dopa (e.g. Ben-Shlomo et al., BMJ 316:1191, 1998).

Overall, the data suggest that deprenyl may or may not have weak effects on longevity.

Several genes in mice have been identified as “longevity” genes because mice with mutations in these genes have greater mean lifespans relative to the expected lifespan of control mice. These genes include the Ames dwarf mutation, and the Snell dwarf mutation. However, these mutations result in small, frail mice which have difficulty feeding. It is believed that the longevity conferred by these mutations is essentially due to calorie restriction. Recent attempts to use gene array analysis, or other genetic screens for genes associated with longevity phenotypes in worms, flies, and rodents have come up with a number of candidate genes. In general, however, they are frequently “stress-response” genes.

Many compounds, such as Gingko, Ginseng, Vitamin C, have been proposed to improve survival, but controlled and statistically significant survival studies reporting the benefit for these compounds are unknown. Vitamin C and a number of drugs reduce the incidence of certain disease conditions, e.g. cardiovascular disease, and so, presumably, would enhance overall longevity.

The inventors of the instant invention have now discovered a surprising use of carboxyfullerenes as agents that promote an increase in the overall length of the expected lifespan of metazoans, including, but not limited to vertebrates.

Other uses for carboxyfullerenes are disclosed in U.S. Pat. No. 6,265,443, issued Jul. 24, 2001 to Choi et al. which is incorporated herein by reference in its entirety.

### SUMMARY OF THE INVENTION

It is in view of the above problems that the present invention was developed.

An embodiment of the instant invention comprises the administration of a composition to metazoans with the result of increasing the metazoan's lifespan, said composition comprising a carboxylated derivative of a C₆₀ fullerene (“carboxyfullerene”), such as a C₆₀ compound having x pairs of adjacent carbon atoms bonded to a pendant carbon wherein said pendant carbon atom is further bonded to two groups of the general formula —COOH and —R, wherein R is independently selected from the group consisting of —COOH and —H, and wherein x is at least 1.

Another embodiment of a compound useful in the composition of the instant invention can be described by the general formula C₆₀ [(CHCOOH)]ₓ[C(COOH)₂]_{y}, wherein x is an integer from 0 to 3, y is an integer from 1 to 4 and x plus y is an integer from 2 to 4.

An additional embodiment of the instant invention is a process for extending a metazoan's expected lifespan by administering a superoxide dismutase-mimetic as well as a composition comprising a superoxide dismutase-mimetic. Likewise, an additional embodiment of the instant invention is a process for extending a metazoan's lifespan by administering a catalase-mimetic as well as a composition comprising a catalase-mimetic.

Another embodiment of the instant invention is a method of treating a metazoan comprising administering an antioxidant to the metazoan, which effectively increases the lifespan of that metazoan or the cells of that metazoan.

Yet an additional embodiment of the instant invention comprises a pharmaceutical composition comprising carboxyfullerenes useful to increase a metazoan's lifespan, wherein said carboxyfullerene comprises a C₆₀ compound having x pairs of adjacent carbon atoms bonded to a pendant carbon atom, wherein said pendant carbon atom is further bonded to two groups of the general formula —COOH and —R, wherein R is independently selected from the group consisting of —COOH and —H, and wherein x is at least 1.

A preferred embodiment of the instant invention is the administration of a C₃ tris malonic acid C₆₀ (“C₃”) to a metazoan to increase that metazoan's lifespan.

Still, a further embodiment of the instant invention comprises a non-metal containing composition which can catalytically eliminate two biologically reactive species. The embodiment is further drawn to catalysts useful in the elimination of reactive oxygen species, especially reactive oxygen species that are physiologically relevant, such as hydrogen peroxide (H₂O₂) and superoxide (O₂—¹⁰⁸ ). The catalyst comprises a malonic acid moiety and does not require a metal or metal ion for catalytic activity. Preferably the catalyst further comprises a fullerene moiety, such as C₆₀ fullerene commonly known as buckminsterfullerene (see FIGS. 1-3 for nonlimiting examples of C₆₀ fullerenes and malonic acid derivatives thereof). More preferably the catalyst comprises a C₆₀ compound having x pairs of adjacent carbon atoms bonded to a pendant carbon, wherein said pendant carbon atom is further bonded to two groups of the general formula —COOH and —R, wherein R is independently selected from the group consisting of —COOH and —H, and wherein x is at least 1.

In yet another embodiment, the invention is drawn to methods of enhancing the elimination of reactive oxygen species in any eukaryotic cell by contacting the cell with a superoxide dismutase mimetic. By using the phrase “enhancing the elimination of reactive oxygen species” the inventors mean that the disclosed composition functions as superoxide dismutase mimetics and/or catalase mimetics and therefore acts to reduce the level of reactive oxygen species in a cell relative to the level of reactive oxygen species in a similar cell that has not been subjected to the disclosed composition. Preferably, the superoxide dismutase or catalase is a catalyst comprising a fullerene moiety. More preferably the catalyst comprises a C₆₀ fullerene having x pairs of adjacent carbon atoms that are bonded to a pendant carbon atom, wherein said pendant carbon atom is further bonded to two groups of the general formula —COOH and —R, wherein R is independently selected from the group consisting of —COOH and —H, and wherein x is at least 1. Most preferably the catalyst comprises C₆₀[C(COOH)₂]₃. By use of the term “C₆₀[C(COOH)₂]₃” throughout the instant application, it is meant that C60 is equivalent to the adjacent carbon atoms of the fullerene, and C is the pendant carbon, which is further bonded to two COOH groups. Reactive oxygen species may be any and all chemicals that are free radicals or contribute to the generation of free radicals, especially physiologically relevant reactive oxygen species that include hydrogen peroxide, superoxide anion, and the like.

It is believed this invention provides a substantial improvement over calorie restriction as a method which substantially increases the lifespan of metazoans, especially humans, given the inherent difficulties within calorie restriction (including, but not limited to, severe limits to food intake as well as the impracticability of use with humans in general).

### BRIEF DESCRIPTION OF THE DRAWINGS

The accompanying drawings, which are incorporated in and form a part of the specification, illustrate the embodiments of the present invention and together with the description, serve to explain the principles of the invention. In the drawings:

FIG. 1 discloses an analysis of C₃ preparations by HPLC identifying three major components all useful in the instant invention.

FIG. 2 displays various carboxyfullerenes useful in the instant invention.

FIG. 3 displays the D3 tris malonic acid regioisomer useful in the instant invention.

FIG. 4 depicts the survival curve for mice treated by the process of the instant invention vs. control.

FIG. 5 illustrates the kinetic analysis of the reaction of superoxide with C_{3.}

FIG. 6 illustrates a characterization of the C₃ molecule following reaction with H₂O₂.

FIG. 7 illustrates the inhibition of pyrogallol autoxidation by C₃.

FIG. 8 illustrates a characterization of the C₃ molecule following reaction with superoxide.

### DETAILED DESCRIPTION OF THE PREFERRED EMBODIMENTS

Referring to the accompanying drawings in which like reference numbers indicate like elements:

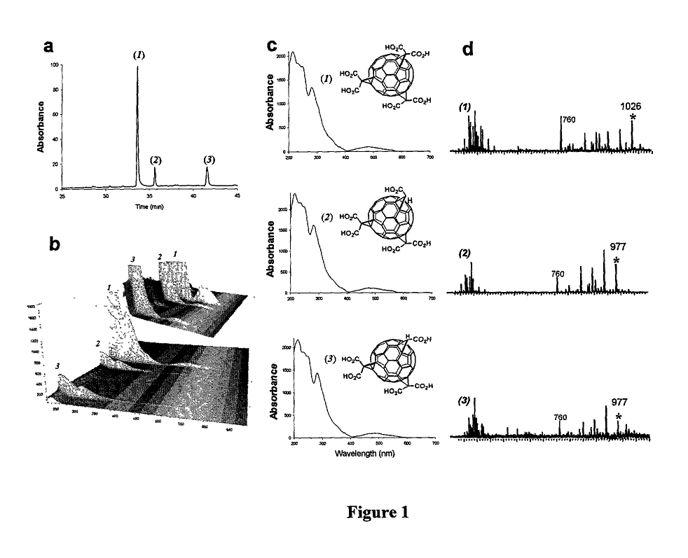

FIG. 1a discloses an analysis of C₃ preparations by HPLC identifying three major components (>99% of the total), all useful in the instant invention.

FIGS. 1b, c indicate all three of the peaks had absorbance spectra characteristic of e, e, e (C₃) additions to the C₆₀ nucleus indicating that the component peaks of C₃ represented e, e, e regioisomers with different headgroups attached to the cyclopropane carbons on C₆₀.

FIGS. 1c(1)-1c(3) show compounds 1-3, after separation of the peaks by HPLC by mass spectrometry as hexacarboxylic acid C₃ (1, 80%) and two isomeric pentacarboxylic acids (2 and 3, 10% each).

FIGS. 1d(1)-1d(3) depict a proton NMR spectroscopy performed with the hexa isomer (1) having no resonance between 4.0 and 6.0 ppm. The first pentacarboxylic acid (2) having a singlet at 4.552 ppm and the second pentacarboxylic acid (3) had a singlet at 4.745 ppm. Based on the NMR results, elution order off the HPLC, and yields (3>2), structures for 2 and 3 were assigned (FIG. 1C).

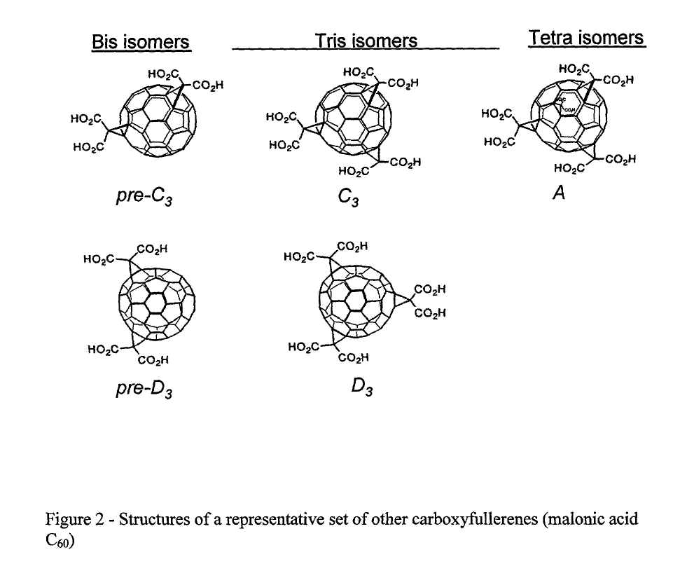

FIG. 2 displays various carboxyfullerenes useful in the instant invention, including 2 bis isomers, 2 tris isomers and a tetra isomer.

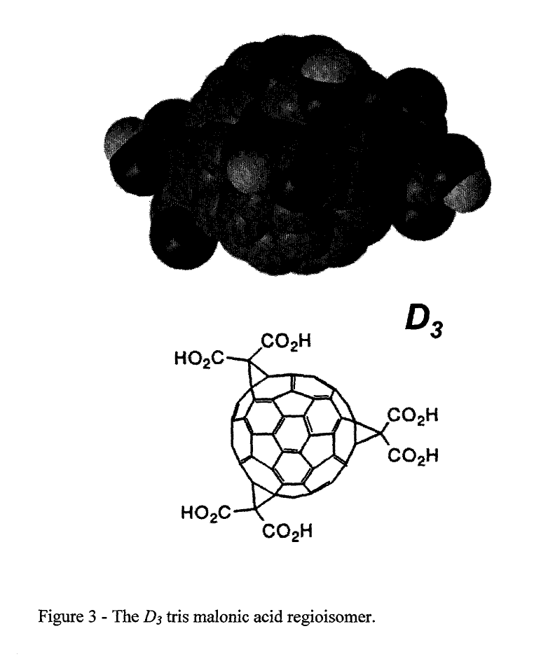

FIG. 3 displays the D3 tris malonic acid regioisomer as both a space filling structure and a chemical structure.

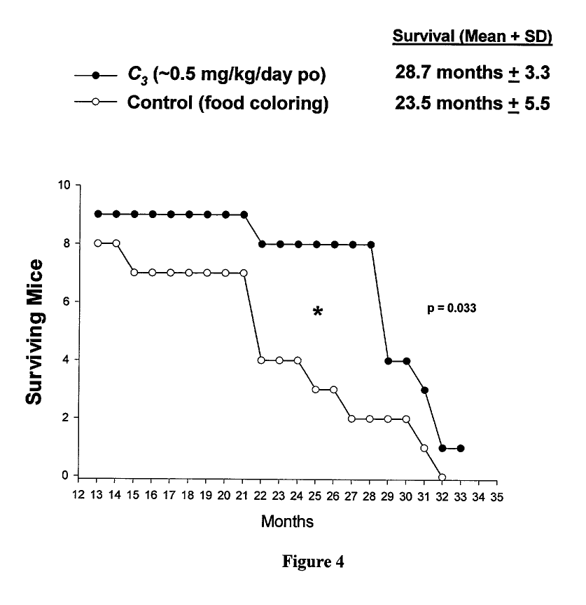

FIG. 4 illustrates the survival curve for mice treated by the process of the instant invention vs. control. FIG. 4 is a Kaplan-Meier survival curve showing lifespan of C57B6 mice treated with either food coloring (control) or C₃ (0.5 mg/kg/day) in their drinking water from the age of 12 months through the end of life. The date of spontaneous death for each mouse was recorded, and used to calculate the lifespan. Lifespan of each mouse was calculated in months because mice received from the NIA rodent colony have their birth month, but not their specific birth date, recorded. Average survival was calculated for each treatment and the mean lifespans were compared using a t-test with significance set to p<0.05 (actual p=0.033). The lifespan of control mice was 23.5±5.5 months (mean±SD, n=8) and of C₃-treated mice was 28.7±3.4 (mean±SD, n=9). This calculation includes one C₃-treated mouse which is still alive as of Feb. 21, 2002, and whose lifespan was included as of this date. Continued survival by this mouse will further increase the average difference in lifespan, and will decrease the p value (increase the significance). Weights of the two groups at 19 months did not differ significantly (in grams): Males 35±4 (control), 35±6 (C₃), Females 27±1 (control), 29+1 (C₃).

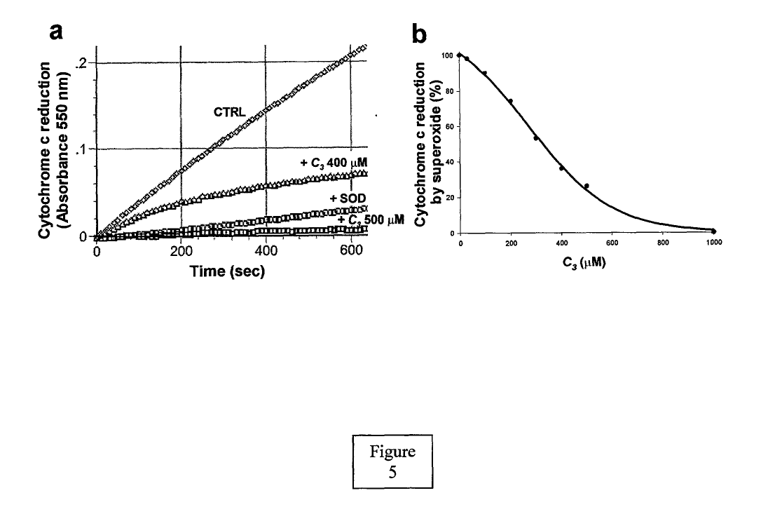

FIG. 5 illustrates the kinetic analysis of the reaction of superoxide with C₃. (a) Superoxide was generated by oxidation of hypoxanthine by xanthine oxidase and assayed by reduction of cytochrome c (at absorbance maximum 550 μm). The rate of cytochrome c reduction was determined for the control reaction (CTRL), and the reaction plus SOD (500 U/ml) or C₃ (400, 500 μM). (b) Graph of the concentration-dependence of superoxide elimination by C₃.

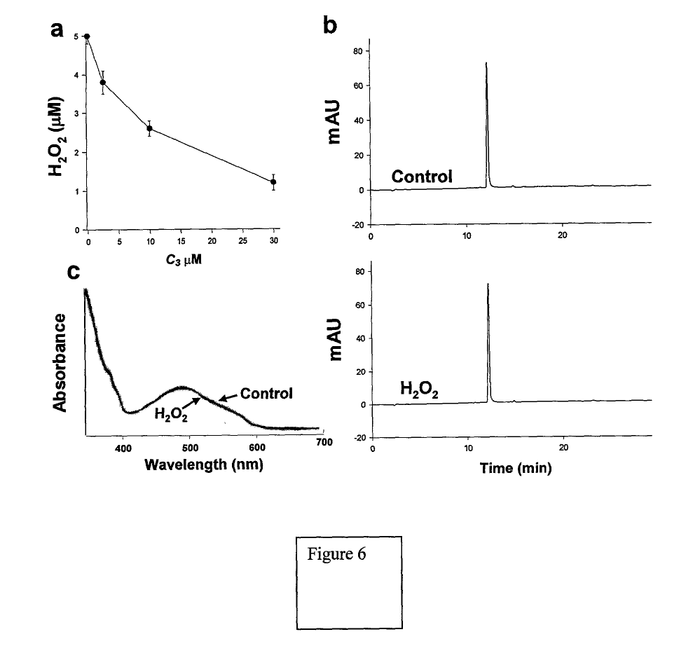

FIG. 6 illustrates a characterization of the C₃ molecule following reaction with H₂O₂. (a) Decomposition of H₂O₂ by C₃, using a fluorescent assay for H₂O₂ (b) Comparison of HPLC chromatograms of C₃ before and after exposure to H₂O₂ (20 mM) show no change in the size or distribution of peaks after exposure to H₂O₂. The absorbance spectrum of C₃ exposed to H₂O₂ was also not altered.

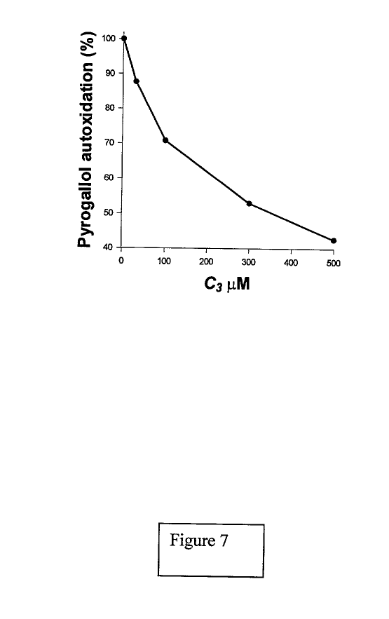

FIG. 7 illustrates the inhibition of pyrogallol autoxidation by C₃. Autoxidation of pyrogallol to purpurogallin, which occurs via a superoxide-dependent mechanism, was decreased by C₃, with an IC₅₀ of 360 μM.

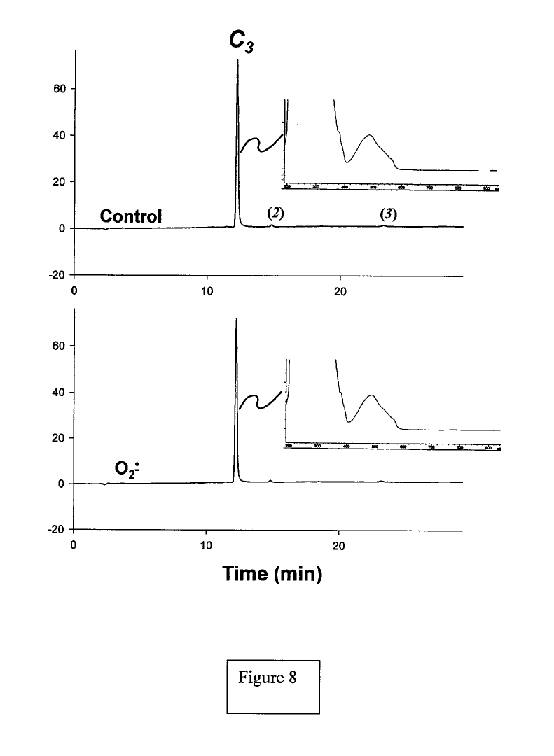

FIG. 8 illustrates a characterization of the C₃ molecule following reaction with superoxide. C₃ was exposed to either superoxide or control solution for 30 min. Samples were then immediately injected onto the HPLC. Chromatograms show a large peak at 12 minutes, representing C₃, and peaks at 14.5 min and 24 min, representing decarboxylation products 1 and 2, respectively. No change in the size or distribution of peaks after exposure to superoxide was seen.

### DETAILED DESCRIPTION OF THE INVENTION

Many important biological reactions generate reactive oxygen species intentionally, or as unwanted toxic by-products. While reactive oxygen species, including superoxide (O₂ [symbol] ) and hydrogen peroxide (H₂O₂), are harnessed for specific physiological functions, they also pose an ongoing threat to the viability and integrity of cells and tissues. In response, cells and organisms have developed a variety of mechanisms to defend themselves against O₂ [symbol] and H₂O₂. In metazoans, O₂ [symbol] is removed by two metallo-enzymes, Cu, Zn-superoxide dismutase (SOD1), and MnSOD (SOD2). H₂O₂, in turn, is removed by catalase, a heme iron containing metallo-enzyme, or glutathione peroxidase, a family of proteins which utilize selenocysteines in conjunction with glutathione to convert H₂O₂ to O₂ and H₂O. However, these endogenous antioxidant defense systems may be overwhelmed under pathological conditions. This has led to attempts to develop additional antioxidants (useful substances that inhibit oxidation or inhibit reactions promoted by oxygen or peroxides) as small molecules to supplement the antioxidant defenses of cells as potential therapeutic agents.

A number of water-soluble C₆₀ derivatives (superoxide dismutase-mimetics) have been found to retain the antioxidant properties of their parent fullerene molecule, allowing its free radical scavenging abilities to be exploited in biological systems and thereby act as agents which reduce cell damage and death.

One group of C₆₀ derivatives, carboxyfullerenes, act as a decomposition catalyst for H₂O₂ and O₂ [symbol] . Although, manganese-containing protoporphyrin compounds, including MnTMPyp, have been reported to act as decomposition catalysts for O₂ [symbol] /H₂O₂, these compounds rely on oxidation-reduction of the manganese atom to catalyze decomposition. It has been now discovered by the inventors that although the C₃ derivative of buckminsterfullerene C₆₀ is a non-metallic compound, it too possesses similar catalytic properties. It is believed this compound is the first non-metallic compound to act in such a manner.

The instant invention utilizes methods of increasing a metazoan's expected lifespan by administering therapeutically effective amounts of antioxidants which result in an extended metazoan, or metazoan's cell, lifespan. In particular, the instant invention utilizes a composition comprising the antioxidant carboxyfullerenes as a treatment to increase the lifespan of metazoans or metazoan cells.

Buckminsterfullerene, C₆₀, is a carbon sphere with alternating 5- and 6-carbon rings; the 30 carbon double bonds react easily with oxygen radicals (Krusic et al, 1991) and so can act as a free radical scavenger. Native C₆₀, however, is soluble only in a limited number of solvents, such as toluene or benzene. To be useful in accordance with the instant invention, C₆₀ compounds of the instant invention, which are referred to as carboxyfullerenes, have been mono- or multiply-derivativized with malonic acid, or the pharmaceutically acceptable malonic acid salts, esters and amides, where the methylene group of the malonic acid is bonded to two carbons of the fullerene sphere. The compounds useful in accordance with the present invention are thus C₆₀ compounds, their corresponding salts, esters and amides having x pairs of adjacent carbon atoms of the C₆₀ fullerene bonded to at least one pendant carbon, wherein the pendant carbon atom is further bonded to two groups of the general formula —COOH and —R, wherein R is independently selected from the group consisting of —COOH and —H, and wherein x is at least 1. Examples of isomers of this general formula are shown in FIGS. 1-3. The preferred compound useful in accordance with the present invention is C₆₀ (C(COOH)₂)₃ and its pharmaceutically acceptable salts, esters and amides.

Thus, the present invention comprises a method of extending the expected lifespan of metazoans or metazoan cells by administering to the metazoan a composition comprising a C₆₀ compound having x pairs of adjacent carbon atoms bonded to a pendant carbon atom, wherein said pendant carbon atom is further bonded to two groups of the general formula —COOH and —R, wherein R is independently selected from the group consisting of —COOH and —H, and wherein x is at least 1. Further, a preferred embodiment of the instant invention comprises C₆₀[(CHCOOH)]ₓ[C(COOH)₂]_{y} compounds, wherein x is a number from 0 to 3, y is a number from 1 to 4 and the sum of x and y is 2 to 4.

The carboxyfullerene compounds of the instant invention can be administered systematically as a composition containing the active compound and a pharmaceutically acceptable carrier compatible with said compound. In preparing such a composition, any conventional pharmaceutically acceptable carrier may be utilized. When the drug is administered orally, it is generally administered at regular intervals.

In therapeutic use, the compounds useful in accordance with the instant invention may be administered by any route whereby drugs are conventionally administered. Such routes include intravenously, intramuscularly, subcutaneously, intrathecally, intraperitoneally, topically, as well as orally. Preferably, the method of the invention is carried out via oral or intravenous routes of administration.

Pharmaceutical compositions utilizing the instant invention can be made up in any conventional form, including a solid form for oral administration such as tablets, capsules, pills, powders, granules, and the like. The pharmaceutical compositions may be sterilized and/or may contain adjuvants such as preservatives, stabilizers, wetting agents, emulsifiers, salts for varying the osmotic pressure, and/or buffers.

Typical preparations for intravenous administration would be sterile aqueous solutions including water/buffered solutions. Intravenous vehicles include fluid, nutrient and electrolyte replenishers. Preservatives and other additives may also be present such as antibiotics and antioxidants.

In accordance with this invention, the carboxyfullerenes described herein are useful in pharmaceutically acceptable oral modes. These pharmaceutical compositions contain said compound in association with a compatible pharmaceutically acceptable carrier material. Any conventional carrier material can be utilized. Any conventional oral dosage form such as tablets, capsules, pills, powders, granules, and the like may be used. The carrier material can be an organic or inorganic inert carrier material suitable for oral administration. Suitable carriers include water, gelatin, gum arabic, lactose, starch, magnesium stearate, talc, vegetable oils, polyalkylene-glycols, petroleum jelly and the like. Furthermore, the pharmaceutical composition may contain other pharmaceutically active agents. Additional additives such as flavoring agents, preservatives, stabilizers, emulsifying agents, buffers and the like may be added in accordance with accepted practices of pharmaceutical compounding.

A preferred oral dosage form comprises tablets, capsules of hard or soft gelatin, methylcellulose or of another suitable material easily dissolved in the digestive tract. The oral dosages contemplated in accordance with the present invention will vary in accordance with the needs of the individual patient as determined by the prescribing physician. The preferred oral dosage form is capsules or tablets containing from 50 to 500 mg of a carboxyfullerene useful in accordance with the present invention.

In carrying out the method of the invention, a compound useful in accordance with the invention can generally be given to adults daily, preferably orally, intramuscularly, subcutaneously or intravenously. If intramuscularly, intravenously or subcutaneously, the instant invention should be given in an amount from as low as about 0.1 mg/kg to an amount as high as 3 mg/kg, with the precise dosage being varied depending upon the needs of the patient. The daily dose, if given orally, would be expected to be as little as 0.1 mg/kg to an amount as high as 15 mg/kg. In general, this therapy may be carried out prophylactically for an indefinite time.

Carboxyfullerene would be administered chronically (e.g. daily) or frequently (e.g. once a week). The C₃ isomer is expected to be the most effective agent. The expected daily dose of the C₃ isomer, if given by intravenous, intramuscular or subcutaneous delivery, would be about 0.1 mg/kg to about 3 mg/kg. The daily does if given orally would be expected to range between 0.1 mg/kg and 15 mg/kg.

The above dosing information is based on a pharmokinetics study carried out in mice, toxicity testing in mice and toxicity testing in rats. In mice, the plasma half-life of C₃ was calculated to be 8 hours. The 50% lethal dose (LD50) for a single injection of C₃ was >70 mg/kg, and C₃ was cleared from mice by excretion through both the liver and kidney. Using calculations based on the pharmacokinetic data, the therapeutic plasma levels appear to be between 0.1 and 1 μg/ml. Although equivalent amounts of C₃ are absorbed if the compound is given by intravenous, intraperitoneal or subcutaneous administrations, only about 1/15^{th} of this dose is absorbed when given orally (e.g. in drinking water). However, the standard pharmaceutical formulations of C₃ for oral delivery are expected to significantly increase the bioavailability of orally-administered C₃ (e.g. incorporation of C₃ into time-release tablets).

It is envisioned that the instant invention is useful for all metazoans, including but not limited to vertebrates, and more specifically to mammals, including humans and their companion animals.

“Lifespan” or “expected lifespan”, as utilized in this patent application, is the average expected length of life of a kind of organism or cell in a particular environment. The lifespan increased by the instant invention is the expected average length of time (from birth to death) that a metazoan would be expected to live (i.e., “generic” expected lifespan), if that metazoan were not utilizing the process of the instant invention. As the results of Example 2 indicate, mice subject to the process of the instant invention had an actual lifespan of 28.7 months, which corresponded to a lifespan that is 20% greater than the control mouse's lifespan of 23.5 months. The lifespan of the control mouse used in this example represents the generic “expected lifespan”.

Because many important biological reactions generate reactive oxygen species intentionally, or as unwanted toxic by-products, antioxidant molecules capable of supplementing the antioxidant defenses of cells as potential therapeutic agents are therapeutically useful.

The compositions of the instant invention have novel antioxidant properties. According to the present invention, the reactivity of the C₃ malonic acid derivative (e,e,e C₆1[C(COOH)₂]₃) with O₂ [symbol] and H₂O₂ was characterized. The Kᵢ of C₃ for O₂ [symbol] was calculated to be 3×10⁶ M⁻¹ sec⁻¹. Analysis of the C₃ molecule after interaction with O₂ [symbol] and H₂O₂ indicated that no permanent chemical or structural changes occurred at either the C₆₀ moiety or the malonic acid groups, supporting the claim that the C₃ molecule is a true catalyst. Although, manganese-containing protoporphyrin and salen compounds have also been reported to act as catalysts for the decomposition of O₂ [symbol] /H₂O₂, these compounds rely on oxidation-reduction of the metal atom to catalyze decomposition, whereas the malonic acid fullerene derivatives of the instant invention do not require a metal atom to catalyze the decomposition of reactive oxygen species.

Thus, in view of the foregoing discovery, the invention is drawn to catalysts useful in the elimination of reactive oxygen species, especially reactive oxygen species that are physiologically relevant, such as hydrogen peroxide (H₂O₂) and superoxide (O₂ [symbol] ). The catalyst comprises a malonic acid moiety and does not require a metal or metal ion for catalytic activity. Preferably the catalyst further comprises a fullerene moiety, such as a 60 carbon (“C₆₀”) fullerene commonly known in the art as a “buckminsterfullerene” (see FIGS. 1-3 for examples of C₆₀ fullerenes and malonic acid derivatives thereof). More preferably, the catalyst comprises a C₆₀ fullerene having x pairs of adjacent carbon atoms that are bonded to at least one pendant group corresponding to a formula [C(R)(COOH)], wherein each R may independently be either another COOH group or a H. Most preferably the catalyst comprises C₆₀[C(COOH)₂]₃.

In yet another embodiment, the invention is drawn to methods of enhancing the elimination of reactive oxygen species in any eukaryotic cell by contacting the cell with a superoxide dismutase mimetic. The composition of the instant invention functions as superoxide dismutase mimetic and therefore acts to reduce the level of reactive oxygen species in a cell relative to the level of reactive oxygen species in a similar cell that has not been subjected to the disclosed composition. Preferably, the superoxide dismutase is a catalyst comprises a fullerene moiety, such as a 60 carbon fullerene. More preferably the catalyst comprises a C₆₀ fullerene having x pairs of adjacent carbon atoms that are bonded to at least one pendant group corresponding to a formula [C(R)(COOH)], wherein each R may be independently either another COOH group or a H. Most preferably the catalyst comprises C₆₀[C(COOH)₂]₃. Reactive oxygen species may be any and all chemicals that are free radicals or contribute to the generation of free radicals, especially physiologically relevant reactive oxygen species that include hydrogen peroxide, superoxide anion, and the like.

The instant invention is further important because it is a non-metal containing composition which can catalytically eliminate two biologically reactive species. Catalysts of the instant invention are useful in the elimination of reactive oxygen species, especially reactive oxygen species that are physiologically relevant, such as hydrogen peroxide (H₂O₂) and superoxide (O₂ [symbol] ). The catalyst of the instant invention comprises a malonic acid moiety which does not require a metal or metal ion for catalytic activity. Preferably, the catalyst comprises a fullerene moiety, such as C₆₀ fullerene. More preferably the catalyst comprises a C₆₀ compound having x pairs of adjacent carbon atoms bonded to two carbons of the C₆₀ sphere, wherein said adjacent carbon atom is further bonded to two groups of the general formula —COOH and —R, wherein R is independently selected from the group consisting of —COOH and —H, and wherein x is at least 1.

The instant invention further enhances the elimination of reactive oxygen species in any eukaryotic cell by contacting the cell with a superoxide dismutase mimetic. The composition of the instant invention functions as superoxide dismutase mimetic and therefore acts to reduce the level of reactive oxygen species in a cell relative to the level of reactive oxygen species in a similar cell that has not been subjected to the disclosed composition. Preferably, the superoxide dismutase is a catalyst comprising a fullerene moiety. More preferably the catalyst comprises a C₆₀ fullerene having x pairs of adjacent carbon atoms that are bonded to at least one pendant group corresponding to a formula [C(R)(COOH)], wherein each R may be either another COOH group or a H. Most preferably the catalyst comprises C₆₀[C(COOH)₂]₃. Reactive oxygen species may be any and all chemicals that are free radicals or contribute to the generation of free radicals, especially physiologically relevant reactive oxygen species that include hydrogen peroxide, superoxide anion, and the like.

The data herein demonstrates that the disclosed carboxyfullerenes are a novel class of antioxidants with the unique ability to scavenge multiple oxygen-derived free radicals, and that these compounds have unusual broad and powerful capabilities to extend the lifespan of individuals.

Further features and advantages of the present invention, as well as the structure and operation of various embodiments of the present invention, are described in detail below with reference to the accompanying drawings.

The above disclosure describes several preferred embodiments of the invention, which do not limit the scope of the invention. The skilled artisan in the practice of this invention will recognize other embodiments of this invention that are not overtly disclosed herein. The invention is further illustrated by the examples described below. These examples are meant to illustrate the invention and are not to be interpreted as limiting the scope of the invention.

All of the references and related art cited herein represent a portion of the present state of the art and are therefore incorporated herein in their entirety.

### EXAMPLE 1

### Preparation of C₃ carboxyfullerenes

Materials. Silica gel (Merck grade 9385, 260-400, 60 A) was obtained from Aldrich Chemicals (St. Louis, Mo.). Other reagents were purchased from Sigma Chemical Co. (St. Louis, Mo.) and other standard sources.

The C₃ regioisomer of malonic acid C₆₀ (e,e,e C₆₀[C(COOH)₂]₃) was synthesized by dissolving C₆₀ (720 mg, 1.00 mmol) in toluene at a concentration of 1 mg/ml by stirring overnight. Dimethyl bromomalonate (632.4 mg, 2.69 mmol) was added, followed by 1,8-diazabicyclo (5.4.0) undec-7-ene (DBU, 493 mg, 3.24 mmol). The reaction mixture was stirred for 2 hours, filtered through a pad of silica gel and concentrated in vacuo. The residue was chromatographed on a 450 ml column of silica gel (Merck, 280-400 mesh), starting in toluene. The colored components were separated by adding increasing amounts of ethyl acetate (EtOAc) to the toluene. The C₃ fraction eluted in 5% EtOAc in toluene. Purity of the C₆₀ malonic ester fractions was monitored by TLC and HPLC. The C3 ester (0.25 g, 0.23 mmol) was dissolved in toluene (250 ml) and sparged with nitrogen. Addition of sodium methoxide (2.22 ml of 2.2 M, 4.88 mmol) resulted in a precipitate within minutes. The mixture was stirred at room temperature under nitrogen for one hour. Water (20 ml) was added and the mixture was stirred overnight. All colored products went into the water layer. The layers were separated, and the aqueous layer was chilled and acidified with 20% sulfuric acid (1.32 ml). The solution was extracted with EtOAc three times, resulting in all color going into the organic layer. The organic layer was washed several times with water to extract a yellow contaminant. The EtOAc layer was then evaporated, and the residue 223.2 mg (89% of theoretical) was freeze-dried.

### EXAMPLE 2

### Experimental Method for Longevity Trial with Mice

Twelve month old C57B6NIH male and female mice (equal numbers) were purchased from the National Institute on Aging (NIA) Aging Rodent Colony. Mice shipped from this colony were not selected in any way for health, tumors or other disabilities, and all mice obtained from the colony were subsequently enrolled in the study. Mice were randomly placed in same-sex numbered cages, two per cage, ear-punched for identification, and weighed. Mice were then trained on a rotorod twice per week for three sessions, and were then tested on the rotorod in three sessions to measure motor performance at baseline. Cages were then assigned to receive either treatment A or treatment B by an observer who was blind to what these treatments would be.

Treatment A was a solution of C₃ (28.75 μM) in water, and treatment B was commercial food coloring added to match the red C₃ solution. Solution A or solution B was placed in the water bottles, and solutions were topped-off twice weekly, and filtered to remove any particulates biweekly by an individual blind to the identity of the solutions. At 19 months of age, mice were weighed again, and underwent another round of rotorod training and testing. Mice were allowed to die spontaneously, and their date of death were recorded by the Animal Housing Facility staff as part of the normal operating procedure of the facility. Facility staff believed that animals were on an antibiotic solution, and did not know the purpose of the study. When animals died, the cage number, identity of the animal, and the date of death were recorded on the death notice, which was then sent to the laboratory, where the information was entered into the database.

The results of these experiments are displayed in FIG. 4 and show a marked increase (approximately 20%) in the lifespan of mice. In addition, because longevity was increased by the oral dosing of a drug, it is the first practical method for achieving increased longevity in metazoans. The increased lifespan of C₃-treated mice was not accompanied by a reduction in weight.

Not to be bound by theory, it is envisioned by the inventors that, the benefits of the instant invention could be utilized to extend the lifespan of all metazoans or metazoan cells, because mice are metazoan organisms. Further, one of ordinary skill in the art would recognize that because the benefits of calorie restriction have been shown in all metazoans tested, that the benefits of the instant invention should further carry over for all metazoans, including all vertebrates, as well as mammals and more specifically, to humans.

### EXAMPLE 3

### Toxicity Study Utilizing Rats

Rat toxicity testing of C₃ was also carried out with two strains of rats (Sprague-Dawley and Long-Evans) which received up to 10 mg/kg day for 30 days without showing any toxicity (i.e., decreased survival, impaired grooming or decreased feeding).

### EXAMPLE 4

### Superoxide Decomposition by C₃ Isomers: Cytochrome C Reduction

The ability of C₃ to eliminate superoxide radical was assessed using three separate methods. In the first, O₂ [symbol] was generated by metabolism of hypoxanthine by xanthine oxidase (reaction 1). O₂ [symbol] production was determined by following cytochrome c reduction (absorbance max 550 nm) on a microtiter platereader as described (Quick et al., 2000). Stock solutions of cytochrome c were evaluated on a Beckman DU650 spectrophotometer to verify that that cytochrome c stock was fully oxidized prior to use, and to confirm the concentration of the stock. The reaction was run in the presence of superoxide dismutase (bovine SOD1, Sigma) to determine the non-specific reduction of cytochrome c (i.e. the rate of reaction in the absence of O₂ [symbol] ). C₃ (100-700 μM) was included to determine the IC₅₀.

To determine whether C₃ directly inhibits xanthine oxidase activity, purine metabolism and oxygen utilization was evaluated in the presence and absence of C₃. Hypoxanthine and its metabolites xanthine and uric acid were analyzed by HPLC at specific times during the xanthine oxidase: hypoxanthine reaction. The reaction was allowed to run for 40 or 100 minutes and was stopped by the addition of trifloroacetic acid (0.1% TFA) to denature xanthine oxidase. Samples were injected onto the HPLC and separated on a C₁₈ column. Concentrations of the purines were compared with those in reactions run with C₃ (100-700 μM) to determine the degree of inhibition by C₃.

The ability of C₃ to eliminate 02! using an alternative method to generate O₂ [symbol] was determined. Pyrogallol autooxidation to purpurogallin was assayed as described. Briefly, Tris-cacodylic acid buffer (55.6 mM) buffer, pH 8.2, was prepared with 1 mM diethyltriaminopentaacetic acid (DTPA) (TCB solution). The pyrogallol solution contained 10 mM pyrogallol in 10 mM HCl. To 0.9 ml of TCB, 0.1 ml of pyrogallol solution was added, rapidly vortexed, and placed in the cuvette. The increase in absorbance at 420 nm was followed for 60 sec in a Beckman DU 650 spectrophotometer at room temperature. The rate of pyrogallol autoxidation in the presence of SOD (100 U/ml) was subtracted from that of the control reaction without SOD to determine the rate that was specifically due to reaction with O₂ [symbol] .

Hydrogen peroxide scavenging was determined using an Amplex red H₂O₂ detection kit (Molecular Probes, Eugene, Oreg.) as per manufacturer's instructions The kit includes horseradish peroxidase (HRP) to catalyze H₂O₂-dependent conversion of resurofin to a fluorescent product, which is followed on a fluorescence plate reader (Bio-Tek FL600).

O₂ consumption during the xanthine oxidase reaction was determined using an oxymetry chamber equipped with a Clark-type O₂ electrode (Hansatech, UK). The effect of superoxide dismutase, or C₃ (0-600 μM), on oxygen consumption was measured.

Results

To define the reaction of C₃ with O₂ [symbol] , two alternative techniques were employed: 1) kinetic analysis of ferricytochrome c reduction by superoxide, and 2) inhibition of pyrogallol autooxidation. Cytochrome c reduction by O₂ [symbol] in the absence or the presence of hexacarboxylic C₃ and the two pentacarboxylic C₃ acids was examined to determine the rate at which each compound could eliminate superoxide anion (FIG. 5). The IC₅₀ for the C₃ mixture (C₃ plus the two decarboxylation products) was 330 μM. The IC₅₀ for the pure hexa acid (1) was 490 μM, and for penta-1 (2) and penta-2 (3) was 300 μM and 230 μM, respectively (Table 1). Under the same assay conditions, the IC₅₀ was 0.5 μM for SOD, and was 50 μM for the metal-dependent SOD mimetic, MnTBAP.

TABLE 1
Reaction kinetics of C₃ with superoxide (O₂)

Cytochrome c reduction

| | | Reaction rate (k) |
|---|---|---|
| Compound | IC50 (μM) | (M-1 sec-1) |
| C₃ mixture (95% hexa acid) | 330 | 3 × 10⁶ |
| C₃ hexacarboxylic acid (1) | 400 | 2.5 × 10⁶ |
| Decarboxylation product (2) | 300 | 3 × 10⁶ |
| Decarboxylation product (3) | 230 | 4 × 10⁶ |
| SOD | 0.5 | 2 × 10⁹ |

It was further determined that C₃ inhibited xanthine oxidase metabolism of hypoxanthine by <10% at 300 μM and 15% at 500 μM, a concentration of C₃ that reduces the concentration of superoxide in the reaction to zero, indicating that the C₃-dependent reduction in superoxide concentration was not due simply to inhibition of the xanthine oxidase reaction. Oxypurinol, a xanthine oxidase inhibitor, however, was able to completely inhibit purine metabolism at concentrations similar to those previously reported (data not shown). Oxygen consumption during the xanthine oxidase reaction was also evaluated with or without the C₃ compounds as a second means of confirming that xanthine oxidase was not inhibited by C₃. Oxygen utilization by xanthine oxidase was similar regardless of whether the reaction was run in the presence of SOD, which does not inhibit xanthine oxidase, or C₃ (FIG. 6). This provides additional evidence that purine oxidation by xanthine oxidase is not affected by C₃.

C₃ and decarboxylation products were also evaluated using an alternative method to generate superoxide, autoxidation of pyrogallol (FIG. 7). In this second assay, the IC₅₀ for the C₃ (mixture) was determined to be approximately 360 μM.

To determine whether elimination of O₂ [symbol] by C₃ was associated with changes in its structure, C₃ compounds were extracted after exposure to superoxide. To maximize the amount of modified C₃ present should structural changes occur, hexacarboxylic acid C₃ and its decarboxylation products were exposed to superoxide generated by the xanthine oxidase reaction for four consecutive reactions by re-addition of the substrate, hypoxanthine. According to the extinction coefficient for reduced cytochrome c (2.9×10⁴ M⁻¹) multiplied by the absorbance at 550 nm produced by the control reaction of hypoxanthine with xanthine oxidase (0.5 OD)=0.172×10⁴ M superoxide, approximately 12% of C₃ should have interacted with one molecule of superoxide. The IC₅₀ concentration (330 μM) eliminated half this concentration, e.g. 0.09×10⁴ M/330×10⁻⁶ μM C₃. Thus, about 3% of C₃ interacts with superoxide per reaction for each of four reactions. Compounds were extracted from the reaction mixture, and re-injected onto the HPLC. Compounds exposed to all the components of the reaction mixture except hypoxanthine (and hence, no superoxide) were also extracted and analyzed. No additional peaks that could represent oxidation products were observed (FIG. 8). Peaks were collected and evaluated by mass spectrometry, but no changes in the molecular ions were observed.

The ability of C₃ to eliminate H₂O₂ was also determined. The IC₅₀ for the C₃ mixture was 1001M. Oxygen consumption studies were performed with H₂O₂ plus catalase or plus C₃. Decomposition of H₂O₂ by catalase resulted in the expected rapid re-evolution of oxygen. C₃ also caused re-evolution of oxygen from H₂O₂, although the rate was significantly slower than that seen with catalase. No change in the C₃ molecule was observed after exposure to even extremely high concentrations of H₂O₂ (20-200 mM), as determined by HPLC and spectroscopy.

In view of the foregoing, it will be seen that the several advantages of the invention are achieved and attained.

The embodiments were chosen and described in order to best explain the principles of the invention and its practical application to thereby enable others skilled in the art to best utilize the invention in various embodiments and with various modifications as are suited to the particular use contemplated.

As various modifications could be made in the constructions and methods herein described and illustrated without departing from the scope of the invention, it is intended that all matter contained in the foregoing description or shown in the accompanying drawings shall be interpreted as illustrative rather than limiting. For example, the process as described above could easily be applied to other metazoans, including but not limited to humans, with the same results. Thus, the breadth and scope of the present invention should not be limited by any of the above-described exemplary embodiments, but should be defined only in accordance with the following claims appended hereto and their equivalents.

What is claimed is:

1. A process for extending the lifespan of a metazoan or metazoan cells comprising administering to said metazoan a composition comprising a C₆₀ compound having x pairs of adjacent carbon atoms bonded to two carbons of said C₆₀ compound wherein said adjacent carbon atom is further bonded to two groups of a general formula —COOH and —R, wherein R is independently selected from the group consisting of —COOH and —H, and wherein x is at least 1.

2. The process of claim 1 where x is 4.

3. The process of claim 2 wherein said composition comprises said C₆₀ compound, its pharmaceutically acceptable salts and pharmaceutically accepted esters, and a pharmaceutically acceptable carrier, present in said composition in a therapeutically effective amount.

4. The process of claim 1 wherein x is 3.

5. The process of claim 4 wherein said C₆₀ compound is C₃ tris malonic acid C₆₀.

6. The process of claim 1 wherein said C₆₀ compound is administered intravenously, intramuscularly, subcutaneously or orally.

7. The process of claim 6 wherein said C₆₀ compound is administered intravenously, intramuscularly or subcutaneously in an amount of at least 0.1 mg/kg.

8. The process of claim 7 wherein said C₆₀ compound is administered intravenously, intramuscularly or subcutaneously in an amount of about 3 mg/kg.

9. The process of claim 6 wherein said C₆₀ compound is administered orally in an amount of at least 0.1 mg/kg.

10. The process of claim 6 wherein said C₆₀ compound is administered orally in an amount of about 15 mg/kg.

11. The process of claim 7 wherein said C₆₀ compound is administered daily.

12. The process of claim 9 wherein said C₆₀ compound is administered daily.

13. The process of claim 1 wherein said metazoan is a vertebrate.

14. The process of claim 1 wherein said metazoan is a mammal.

15. The process of claim 1 wherein said metazoan is a human.

16. A process for extending a metazoan's lifespan comprising regularly administering a superoxide dismutase-mimetic to said metazoan wherein said metazoan's lifespan is extended.

17. The process of claim 16 wherein said superoxide dismutase-mimetic comprises a non-metallic compound.

18. The process of claim 17 wherein said superoxide dismutase-mimetic comprises a carboxyfullerene.

19. The process of claim 18 wherein said carboxyfullerene comprises a C₆₀ compound having x pairs of adjacent carbon atoms bonded to two carbons of said C₆₀ compound wherein said adjacent carbon atom is further bonded to two groups of a general formula —COOH and —R, wherein R is independently selected from the group consisting of —COOH and —H, and wherein x is at least 1.

20. The process of claim 19 wherein x is about 4.

21. The process of claim 19 wherein x is 3.

22. The process of claim 21 wherein said C₆₀ compound is C₃ tris malonic acid C₆₀.

23. The process of claim 18 wherein said carboxyfullerene is administered intravenously, intramuscularly, subcutaneously or orally.

24. The process of claim 23 wherein said carboxyfullerene is administered intravenously, intramuscularly or subcutaneously in an amount of at least 0.1 mg/kg.

25. The process of claim 24 wherein said carboxyfullerene is administered intravenously, intramuscularly or subcutaneously in an amount of about 3 mg/kg.

26. The process of claim 18 wherein said carboxyfullerene is administered orally in an amount at least 0.1 mg/kg.

27. The process of claim 26 wherein said carboxyfullerene is administered orally in an amount of about 15 mg/kg.

28. The process of claim 24 wherein said compound is administered daily.

29. The process of claim 26 wherein said compound is administered daily.

30. The process of claim 16 wherein said metazoan is a human.

31. A process for extending a metazoan's lifespan comprising regularly administering an antioxidant compound to said metazoan, wherein said compound is introduced into said metazoan intravenously, intramuscularly, subcutaneously or through oral delivery.

32. The process of claim 31 wherein said compound comprises a C₆₀ compound having x pairs of adjacent carbon atoms bonded to two carbons of said C₆₀ compound wherein said adjacent carbon atom is further bonded to two groups of a general formula —COOH and —R, wherein R is independently selected from the group consisting of —COOH and —H, and wherein x is at least 1.

33. A pharmaceutical composition useful for extending a metazoan's lifespan, said composition comprising a C₆₀ compound having x pairs of adjacent carbon atoms bonded to two carbons of said C₆₀ compound wherein said adjacent carbon atom is further bonded to two groups of a general formula —COOH and —R, wherein R is independently selected from the group consisting of —COOH and —H, and wherein x is at least 1.

34. The composition of claim 33 wherein said composition comprises said compound, its pharmaceutically acceptable salts and pharmaceutically accepted esters, and a pharmaceutically acceptable carrier, wherein said compound is present in said composition in a therapeutically effective amount.

35. The composition of claim 33 wherein x is about 4.

36. A non-metal-containing composition which can catalytically eliminate two biologically reactive oxygen species.

37. The composition of claim 36 wherein said biologically reactive oxygen species are 02! and H₂O₂.

38. The composition of claim 36 wherein said composition comprises a carboxyfullerene.

39. The composition of claim 38 wherein said carboxyfullerene comprises a C₆₀ compound having x pairs of adjacent carbon atoms bonded to two carbons of said C₆₀ compound wherein said adjacent carbon atom is further bonded to two groups of a general formula —COOH and —R, wherein R is independently selected from the group consisting of —COOH and —H, and wherein x is at least 1.

40. The composition of claim 39 wherein x is about 4.

41. The composition of claim 39 wherein said composition comprises C₃ tris malonic acid C₆₀.

42. A catalyst useful in the elimination of reactive oxygen species, wherein the catalyst (a) comprises a malonic acid moiety and (b) does not comprise a metal.

43. The catalyst of claim 42 further comprising a fullerene moiety.

44. The catalyst of claim 43, wherein the catalyst comprises a C₆₀ compound having x pairs of adjacent carbon atoms bonded to two carbons of said C₆₀ compound wherein said adjacent carbon atom is further bonded to two groups of a general formula —COOH and —R, wherein R is independently selected from the group consisting of —COOH and —H. and wherein x is at least 1.

45. The catalyst of claim 44, wherein x is 3.

46. The catalyst of claim 42, wherein the reactive oxygen species is hydrogen peroxide.

47. The catalyst of claim 42, wherein the reactive oxygen species is a superoxide anion.

48. A method of enhancing the elimination of reactive oxygen species in a cell, comprising contacting a cell with a superoxide dismutase mimetic, wherein the level of reactive oxygen species in the cell decreases as compared to the level of reactive oxygen species in a cell that has not been contacted with the superoxide dismutase mimetic.

49. The method of claim 48 wherein the superoxide dismutase mimetic (a) comprises a malonic acid moiety, (b) comprises a fullerene moiety and (c) does not comprise a metal.

50. The method of claim 49 wherein the superoxide dismutase mimetic comprises a C₆₀ compound having x pairs of adjacent carbon atoms bonded to two carbons of said C₆₀ compound wherein said adjacent carbon atom is further bonded to two groups of a general formula —COOH and —R, wherein R is independently selected from the group consisting of —COOH and —H, and wherein x is at least 1.

51. The method of claim 50 wherein x is equal to 3.

52. The method of claim 48, wherein the cell is a metazoan cell.

53. The method of claim 52, wherein the cell is a human cell.

54. The method of claim 48 wherein the reactive oxygen species is a hydrogen peroxide.

55. The method of claim 48 wherein the reactive oxygen species is a superoxide anion.

56. A process for extending the lifespan of a human comprising administering to said human a composition comprising a C₆₀ compound having x pairs of adjacent carbon atoms bonded to two carbons of said C₆₀ compound wherein said adjacent carbon atom is further bonded to two groups of a general formula —COOH and —R, wherein R is independently selected from the group consisting of —COOH and —H, and wherein x is at least 1.

57. The process of claim 56 where x is about 4.

58. The process of claim 56 wherein said composition comprises said C₆₀ compound, its pharmaceutically acceptable salts and pharmaceutically accepted esters, and a pharmaceutically acceptable carrier, present in said composition in a therapeutically effective amount.

59. The process of claim 56 wherein x is 3.

60. The process of claim 59 wherein said C₆₀ compound is C₃ tris malonic acid C₆₀.

61. The process of claim 56 wherein said C₆₀ compound is administered intravenously, intramuscularly, subcutaneously or orally.

62. The process of claim 61 wherein said C₆₀ compound is administered intravenously, intramuscularly or subcutaneously in an amount of at least 0.1 mg/kg.

63. The process of claim 62 wherein said C₆₀ compound is administered intravenously, intramuscularly or subcutaneously in an amount of about 3 mg/kg.

64. The process of claim 61 wherein said C₆₀ compound is administered orally in an amount of at least 0.1 mg/kg.

65. The process of claim 64 wherein said C₆₀ compound is administered orally in an amount of about 15 mg/kg.

66. The process of claim 62 wherein said C₆₀ compound is administered daily.

67. The process of claim 64 wherein said C₆₀ compound is administered daily.

68. A process for extending a human's lifespan comprising regularly administering an antioxidant compound to said human, wherein said compound is introduced into said human intravenously, intramuscularly, subcutaneously or through oral delivery.

69. The process of claim 68 wherein said compound comprises a C₆₀ compound having x pairs of adjacent carbon atoms bonded to two carbons of said C₆₀ compound wherein said adjacent carbon atom is further bonded to two groups of a general formula —COOH and —R, wherein R is independently selected from the group consisting of —COOH and —H, and wherein x is at least 1.
